(19) United States
(12) Patent Application Publication
(10) Pub. No.: US 2026/0315572 A1
(43) Pub. Date: Oct. 15, 2026

(54) GIP AND GLP-1 DUAL AGONIST COMPOUNDS

(71) Applicant: Eli Lilly and Company, Indianapolis, IN (US)

(72) Inventors: Jorge ALSINA-FERNANDEZ, Indianapolis, IN (US); Robert Andrew BROWN, Carmel, IN (US)

(21) Appl. No.: 19/108,604

(22) PCT Filed: Sep. 14, 2023
(86) PCT No.: PCT/US2023/074128

§ 371 Date: Mar. 4, 2025

Related U.S. Application Data
(60) Provisional application No. 63/406,995, filed on Sep. 15, 2022.

(51) Int. Cl. A61K 38/26; A61K 45/06; A61P 3/04; A61P 3/10

(52) U.S. Cl. CPC A61K 38/26; A61K 45/06; A61P 3/04; A61P 3/10

(57) ABSTRACT

The present invention relates to a pharmaceutical composition comprising a dual incretin peptide that agonizes receptors for both human glucose dependent insulinotropic polypeptide (GIP) and glucagon like peptide 1 (GLP-1).

The present invention relates to the field of medicine. More particularly, the present invention relates to dual incretin peptide mimetic compounds wherein a single peptide agonizes receptors for both human glucose-dependent insulinotropic polypeptide (GIP) and glucagon-like peptide-1 (GLP-1). Such compounds may be useful for treating type 2 diabetes mellitus (T2D).

T2D is the most common form of diabetes accounting for approximately 90% of all diabetes. T2D is characterized by high blood glucose levels caused by insulin-resistance. Additional treatment options for T2D are desired. Compounds offering dual mechanism of action with GIP and GLP 1 are especially desired.

GIP is a 42-amino acid gastrointestinal regulatory peptide that is reported to play a physiological role in glucose homeostasis by stimulating insulin secretion from pancreatic beta cells in the presence of glucose and protecting pancreatic beta cells. GLP-1 is a 37-amino acid peptide that is reported to stimulate insulin secretion, protect pancreatic beta cells, and inhibit glucagon secretion, gastric emptying and food intake leading to weight loss. GIP and GLP-1 are known as incretins; incretin receptor signaling exerts physiologically relevant action critical for glucose homeostasis. In normal physiology, GIP and GLP-1 are secreted from the gut following a meal, and these incretins enhance the physiological response to food including sensation of satiety, insulin secretion, and nutrient disposal. T2D patients have impaired incretin responses.

Glucagon is a 29-amino acid peptide produced by the pancreas, and when bound to glucagon receptor, signals the liver to release glucose leading to an increase in blood glucose. GLP-2, a peptide that like GLP-1 is produced from processing of proglucagon, is known to be associated with cellular proliferation in the gut. Thus, stimulation of glucagon and GLP-2 receptors should be minimized during chronic treatment of T2D patients in order to maximize glucose lowering and reduce potential long term carcinogenic risks.

Certain GIP analogs have been described as exhibiting both GIP and GLP-1 activity in WO 2013/164483, WO 2014/192284, and WO 2011/119657. Further, the compound tirzepatide, a GIP/GLP1 receptor agonist peptide, is described and claimed in U.S. Pat. No. 9,474,780. Tirzepatide is approved by the United States Food and Drug Administration for use in the treatment of diabetes, as an adjunct to diet and exercise.

DPP IV is in the exopeptidase class of proteolytic enzymes. The introduction of non-natural amino acids in a sequence may increase the proteolytic stability of any given peptide against DPP IV degradation.

There remains a need to provide additional treatment options for patients with T2D. The present single peptide compounds are dual agonists with both GIP and GLP-1 receptor agonism. Such peptides may deliver adequate stability against DPP IV and other forms of degradation, but while still maintaining a low immunogenicity potential. Provided are pharmaceutical compositions for use as a therapeutic.

Accordingly, an embodiment of the present invention provides a compound of Formula I:

Y-Aib-EGTFTSDYSI-Aib-LDKIAQ-K((2-[2-(2-Amino-ethoxy)-ethoxy]-acetyl)ₐ-(γ-Glu)_{b}-CO—(CH₂)_{c}—CO₂H)-A-X₂₂-VQWLIAG-X₃₀
wherein
X₂₂ is F or 1-Nal;
X₃₀ is selected from the group consisting of G-NH₂, G-OH, GP-OH, GPSSG-NH₂ (SEQ ID NO: 15) and GPSSG-OH (SEG ID NO: 5);
a is 1 or 2;
b is 1 or 2;
c is 16 or 18;
the C-terminal amino acid is optionally amidated as a C-terminal primary amide (SEQ ID NO: 6); or
a pharmaceutically acceptable salt thereof.

In an embodiment is a compound of Formula I wherein c is 18, or a pharmaceutically acceptable salt thereof.

In an embodiment is a compound of the formula:
Y-Aib-EGTFTSDYSI-Aib-LDKIAQ-K((2-[2-(2-Amino-ethoxy)-ethoxy]-acetyl)ₐ-(γ-Glu)_{b}-CO—(CH₂)_{c}—CO₂H)-A-X₂₂-VQWLIAG-X₃₀ (SEQ ID NO:7)
wherein
X₂₂ is F or 1-Nal;
X₃₀ is selected from the group consisting of G-NH₂, G-OH, GP-OH, GPSSG-NH₂ (SEQ ID NO: 15) or GPSSG-OH (SEQ ID NO:5);
a is 1 or 2;
b is 1 or 2;
c is 16 or 18; or a pharmaceutically acceptable salt thereof.

In an embodiment is a compound selected from the group consisting of

(SEQ ID NO: 8)
Y-Aib-EGTFTSDYSI-Aib-LDKIAQ-K((2-[2-(2-Amino-
ethoxy)-ethoxy]-acetyl)₂-(γ-Glu)-CO-(CH₂)₁₈-
CO₂H)-AFVQWLIAGG-NH₂;
(SEQ ID NO: 9)
Y-Aib-EGTFTSDYSI-Aib-LDKIAQ-K((2-[2-(2-Amino-
ethoxy)-ethoxy]-acetyl)₂-(γ-Glu)-CO-(CH₂)₁₈-
CO₂H)-AFVQWLIAGG-OH;
(SEQ ID NO: 10)
Y-Aib-EGTFTSDYSI-Aib-LDKIAQ-K((2-[2-(2-Amino-
ethoxy)-ethoxy]-acetyl)₂-(γ-Glu)-CO-(CH₂)₁₈-
CO₂H)-AFVQWLIAGGPSSG-NH₂;
(SEQ ID NO: 11)
Y-Aib-EGTFTSDYSI-Aib-LDKIAQ-K((2-[2-(2-Amino-
ethoxy)-ethoxy]-acetyl)₂-(γ-Glu)-CO-(CH₂)₁₈-
CO₂H)-AFVQWLIAGGPSSG-OH;
(SEQ ID NO: 12)
Y-Aib-EGTFTSDYSI-Aib-LDKIAQ-K((2-[2-(2-Amino-
ethoxy)-ethoxy]-acetyl)₂-(γ-Glu)-CO-(CH₂)₁₈-
CO₂H)-AFVQWLIAGGP-OH,
or

a pharmaceutically acceptable salt thereof.

In an embodiment is a pharmaceutical composition comprising a compound of the formula:
Y-Aib-EGTFTSDYSI-Aib-LDKIAQ-K((2-[2-(2-Amino-ethoxy)-ethoxy]-acetyl)ₐ-(γ-Glu)_{b}-CO—(CH₂)_{c}—CO₂H)-A-X₂₂-VQWLIAG-X₃₀
wherein
X₂₂ is F or 1-Nal;
X₃₀ is selected from the group consisting of G, GP, and GPSSG (SEQ ID NO:5);
a is 1 or 2;
b is 1 or 2;
c is 16 or 18;
the C-terminal amino acid is optionally amidated as a C-terminal primary amide (SEQ ID NO: 13); or
a pharmaceutically acceptable salt thereof.

In an embodiment is a pharmaceutical composition comprising a compound of Formula I wherein c is 18, or a pharmaceutically acceptable salt thereof.

In an embodiment is a pharmaceutical composition comprising a compound of the formula:

(SEQ ID NO: 14)
Y-Aib-EGTFTSDYSI-Aib-LDKIAQ-K((2-[2-(2-Amino-
ethoxy)-ethoxy]-acetyl)ₐ-(γ-Glu)_{b}-CO-(CH₂)_{c}-
CO₂H)-A-X₂₂-VQWLIAG-X₃₀

wherein
X₂₂ is F or 1-Nal;
X₃₀ is selected from the group consisting of G, GP, and GPSSG (SEQ ID NO:5);
a is 1 or 2;
b is 1 or 2;
c is 16 or 18; or a pharmaceutically acceptable salt thereof.

In an embodiment is a pharmaceutical composition comprising a compound selected from the group consisting of

(SEQ ID NO: 8)
Y-Aib-EGTFTSDYSI-Aib-LDKIAQ-K((2-[2-(2-Amino-
ethoxy)-ethoxy]-acetyl)₂-(γ-Glu)-CO-(CH₂)₁₈-
CO₂H)-AFVQWLIAGG-NH₂;
(SEQ ID NO: 9)
Y-Aib-EGTFTSDYSI-Aib-LDKIAQ-K((2-[2-(2-Amino-
ethoxy)-ethoxy]-acetyl)₂-(γ-Glu)-CO-(CH₂)₁₈-
CO₂H)-AFVQWLIAGG-OH;
(SEQ ID NO: 10)
Y-Aib-EGTFTSDYSI-Aib-LDKIAQ-K((2-[2-(2-Amino-
ethoxy)-ethoxy]-acetyl)₂-(γ-Glu)-CO-(CH₂)₁₈-
CO2H)-AFVQWLIAGGPSSG-NH₂;
(SEQ ID NO: 11)
Y-Aib-EGTFTSDYSI-Aib-LDKIAQ-K((2-[2-(2-Amino-
ethoxy)-ethoxy]-acetyl)₂-(γ-Glu)-CO-(CH₂)₁₈-
CO₂H)-AFVQWLIAGGPSSG-OH;
(SEQ ID NO: 12)
Y-Aib-EGTFTSDYSI-Aib-LDKIAQ-K((2-[2-(2-Amino-
ethoxy)-ethoxy]-acetyl)₂-(γ-Glu)-CO-(CH₂)₁₈-
CO₂H)-AFVQWLIAGGP-OH,

or a pharmaceutically acceptable salt thereof.

In an embodiment, is a pharmaceutical composition comprising a compound of the formula:
Y-Aib-EGTFTSDYSI-Aib-LDKIAQ-K((2-[2-(2-Amino-ethoxy)-ethoxy]-acetyl)₂-(γ-Glu)-CO—(CH₂)₁₈—CO₂H)-AFVQWLIAGGP-OH; or a pharmaceutically acceptable salt thereof.

In an embodiment, a pharmaceutical composition wherein the pharmaceutical composition comprises a compound of the formula
Y-Aib-EGTFTSDYSI-Aib-LDKIAQ-K((2-[2-(2-Amino-ethoxy)-ethoxy]-acetyl)₂-(γ-Glu)-CO—(CH₂)₁₈—CO₂H)-AFVQWLIAGGP-OH; or
a pharmaceutically acceptable salt thereof; further comprising at least one excipient or diluent.

In an embodiment is a compound of the formula:
Y-Aib-EGTFTSDYSI-Aib-LDKIAQ-K-A-X₂₂-VQWLIAG-X₃₀
wherein
X₂₂ is F or 1-Nal;
X₃₀ is selected from the group consisting of G, GP, and GPSSG;
the C-terminal amino acid is optionally amidated as a C-terminal primary amide; or a pharmaceutically acceptable salt thereof.

In an embodiment is a compound of the formula:
Y-Aib-EGTFTSDYSI-Aib-LDKIAQ-K-A-X₂₂-VQWLIAG-X₃₀
wherein
X₂₂ is F or 1-Nal;
X₃₀ is GP;
the C-terminal amino acid is optionally amidated as a C-terminal primary amide; or a pharmaceutically acceptable salt thereof.

In an embodiment is a pharmaceutical composition comprising a compound of the formula:
Y-Aib-EGTFTSDYSI-Aib-LDKIAQ-K-A-X₂₂-VQWLIAG-X₃₀
wherein
X₂₂ is F or 1-Nal;
X₃₀ is selected from the group consisting of G, GP, and GPSSG;
the C-terminal amino acid is optionally amidated as a C-terminal primary amide; or a pharmaceutically acceptable salt thereof.

In an embodiment is a pharmaceutical composition comprising a compound of the formula:
Y-Aib-EGTFTSDYSI-Aib-LDKIAQ-K-A-X₂₂-VQWLIAG-X₃₀
wherein
X₂₂ is F or 1-Nal;
X₃₀ is selected from the group consisting of G, GP, and GPSSG;
the C-terminal amino acid is optionally amidated as a C-terminal primary amide; or a pharmaceutically acceptable salt thereof, and at least one pharmaceutically acceptable excipient.

In a further embodiment, the present invention provides a compound of Formula I, wherein a is 2, b is 1; and c is 18; or a pharmaceutically acceptable salt thereof.

In an embodiment, the present invention provides a pharmaceutical composition comprising a compound of the Formula:

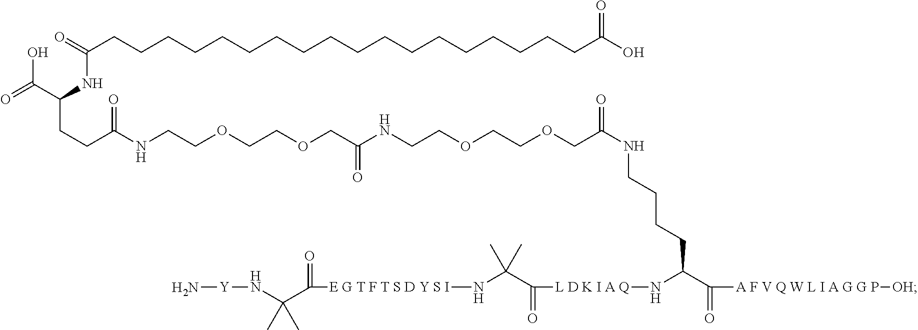

or a pharmaceutical salt thereof.

In an embodiment is a pharmaceutical composition comprising a compound selected from the group consisting of:
Y-Aib-EGTFTSDYSI-Aib-LDKIAQ-K((2-[2-(2-Amino-ethoxy)-ethoxy]-acetyl)₂-(γ-Glu)-CO—(CH₂)₁₈—CO₂H)-AFVQWLIAGGP-NH₂
Y-Aib-EGTFTSDYSI-Aib-LDKIAQ-K((2-[2-(2-Amino-ethoxy)-ethoxy]-acetyl)₂-(γ-Glu)-CO—(CH₂)₁₈—CO₂H)-AFVQWLIAGG-NH₂
Y-Aib-EGTFTSDYSI-Aib-LDKIAQ-K((2-[2-(2-Amino-ethoxy)-ethoxy]-acetyl)₂-(γ-Glu)-CO—(CH₂)₁₈—CO₂H)-AFVQWLIAGG-OH
Y-Aib-EGTFTSDYSI-Aib-LDKIAQ-K((2-[2-(2-Amino-ethoxy)-ethoxy]-acetyl)₂-(γ-Glu)-CO—(CH₂)₁₈—CO₂H)-AFVQWLIAGGPSSG-NH₂
Y-Aib-EGTFTSDYSI-Aib-LDKIAQ-K((2-[2-(2-Amino-ethoxy)-ethoxy]-acetyl)₂-(γ-Glu)-CO—(CH₂)₁₈—CO₂H)-AFVQWLIAGGPSSG-OH; and
Y-Aib-EGTFTSDYSI-Aib-LDKIAQ-K((2-[2-(2-Amino-ethoxy)-ethoxy]-acetyl)₂-(γ-Glu)-CO—(CH₂)₁₈—CO₂H)-AFVQWLIAGGP-OH; or
a pharmaceutically acceptable salt thereof.

In an embodiment, the present invention provides a composition comprising a compound of the formula: Y-Aib-EGTFTSDYSI-Aib-LDKIAQ-K((2-[2-(2-Amino-ethoxy)-ethoxy]-acetyl)ₐ-(γ-Glu)_{b}-CO—(CH₂)_{c}—CO₂H)-A-X₂₂-VQWLIAG-X₃₀
wherein
X₂₂ is F or 1-Nal;
X₃₀ is selected from the group consisting of G, GP, and GPSSG;
a is 1 or 2;
b is 1 or 2;
c is 16 or 18;
the C-terminal amino acid is optionally amidated as a C-terminal primary amide; or
a pharmaceutically acceptable salt thereof.

In an embodiment, the present invention provides a composition comprising a compound of the formula: Y-Aib-EGTFTSDYSI-Aib-LDKIAQ-K((2-[2-(2-Amino-ethoxy)-ethoxy]-acetyl)ₐ-(γ-Glu)_{b}-CO—(CH₂)_{c}—CO₂H)-A-X₂₂-VQWLIAG-X₃₀
wherein
X₂₂ is F or 1-Nal;
X₃₀ is selected from the group consisting of G, GP, and GPSSG;
a is 1 or 2;
b is 1 or 2;
c is 16 or 18;
the C-terminal amino acid is optionally amidated as a C-terminal primary amide; or a pharmaceutically acceptable salt thereof; with a pharmaceutically acceptable carrier, diluent, or excipient.

In an embodiment, the present invention provides a method of treating type 2 diabetes mellitus, comprising administering to a patient in need thereof, an effective amount of a compound of the formula: formula: Y-Aib-EGTFTSDYSI-Aib-LDKIAQ-K((2-[2-(2-Amino-ethoxy)-ethoxy]-acetyl)ₐ-(γ-Glu)_{b}-CO—(CH₂)_{c}—CO₂H)-A-X₂₂-VQWLIAG-X₃₀
wherein
X₂₂ is F or 1-Nal;
X₃₀ is selected from the group consisting of G, GP, and GPSSG;
a is 1 or 2;
b is 1 or 2;
c is 16 or 18;
the C-terminal amino acid is optionally amidated as a C-terminal primary amide; or a pharmaceutically acceptable salt thereof. In a further embodiment, the present invention provides a method of treating type 2 diabetes mellitus comprising administering a compound of the formula: Y-Aib-EGTFTSDYSI-Aib-LDKIAQ-K((2-[2-(2-Amino-ethoxy)-ethoxy]-acetyl)ₐ-(γ-Glu)_{b}-CO—(CH₂)_{c}—CO₂H)-A-X₂₂-VQWLIAG-X₃₀;
wherein
X₂₂ is F or 1-Nal;
X₃₀ is selected from the group consisting of G, GP, and GPSSG;
a is 1 or 2;
b is 1 or 2;
c is 16 or 18;
the C-terminal amino acid is optionally amidated as a C-terminal primary amide; or a pharmaceutically acceptable salt thereof; simultaneously, separately, or sequentially in combination with an effective amount of one or more agents selected from metformin, thiazolidinediones, sulfonylureas, dipeptidyl peptidase 4 inhibitors, and sodium glucose co-transporters. In an embodiment, the present invention provides a method of treating type 2 diabetes mellitus comprising administering a compound of the formula: Y-Aib-EGTFTSDYSI-Aib-LDKIAQ-K((2-[2-(2-Amino-ethoxy)-ethoxy]-acetyl)ₐ-(γ-Glu)_{b}-CO—(CH₂)_{c}—CO₂H)-A-X₂₂-VQWLIAG-X₃₀;
wherein
X₂₂ is F or 1-Nal;
X₃₀ is selected from the group consisting of G, GP, and GPSSG;
a is 1 or 2;
b is 1 or 2;
c is 16 or 18;
the C-terminal amino acid is optionally amidated as a C-terminal primary amide; or a pharmaceutically acceptable salt thereof; simultaneously, separately, or sequentially in combination with an effective amount of one or more agents selected from a GIP/GLP1 agonist, metformin, thiazolidinediones, sulfonylureas, dipeptidyl peptidase 4 inhibitors, and sodium glucose co-transporters. In an embodiment a GIP/GLP1 agonist is tirzepatide or a pharmaceutically acceptable salt thereof.

In an embodiment, the present invention provides a method to improve glycemic control in adults with type 2 diabetes mellitus, comprising administering to a patient in need thereof, an effective amount of a compound of formula: Y-Aib-EGTFTSDYSI-Aib-LDKIAQ-K((2-[2-(2-Amino-ethoxy)-ethoxy]-acetyl)ₐ-(γ-Glu)_{b}-CO—(CH₂)_{c}—CO₂H)-A-X₂₂-VQWLIAG-X₃₀;
wherein
X₂₂ is F or 1-Nal;
X₃₀ is selected from the group consisting of G, GP, and GPSSG;
a is 1 or 2;
b is 1 or 2;
c is 16 or 18;
the C-terminal amino acid is optionally amidated as a C-terminal primary amide; or a pharmaceutically acceptable salt thereof; as an adjunct to diet and exercise. In an embodiment, the present invention provides a method for chronic weight management in adults with an initial body mass index ≥27, comprising administering to a patient in need thereof, an effective amount of a compound of the formula: Y-Aib-EGTFTSDYSI-Aib-LDKIAQ-K((2-[2-(2-Amino-ethoxy)-ethoxy]-acetyl)ₐ-(γ-Glu)_{b}-CO—(CH₂)_{c}—CO₂H)-A-X₂₂-VQWLIAG-X₃₀
wherein
X₂₂ is F or 1-Nal;
X₃₀ is selected from the group consisting of G, GP, and GPSSG;
a is 1 or 2;
b is 1 or 2;
c is 16 or 18;
the C-terminal amino acid is optionally amidated as a C-terminal primary amide; or a pharmaceutically acceptable salt thereof; as an adjunct to a reduced-calorie diet and increased physical activity.

In an embodiment, the present invention provides a method for chronic weight management in adults with an initial body mass index ≥27, comprising administering to a patient in need thereof, an effective amount of a compound of the formula: Y-Aib-EGTFTSDYSI-Aib-LDKIAQ-K((2-[2-(2-Amino-ethoxy)-ethoxy]-acetyl)ₐ-(γ-Glu)_{b}-CO—(CH₂)_{c}—CO₂H)-A-X₂₂-VQWLIAG-X₃₀
wherein
X₂₂ is F or 1-Nal;
X₃₀ is selected from the group consisting of G, GP, and GPSSG;
a is 1 or 2;
b is 1 or 2;
c is 16 or 18;
the C-terminal amino acid is optionally amidated as a C-terminal primary amide; or
a pharmaceutically acceptable salt thereof; simultaneously, separately, or sequentially in combination with an effective amount of one or more agents selected from a GIP/GLP1 agonist, metformin, thiazolidinediones, sulfonylureas, dipeptidyl peptidase 4 inhibitors, and sodium glucose co-transporters. In an embodiment a GIP/GLP1 agonist is tirzepatide or a pharmaceutically acceptable salt thereof.

In an embodiment, the present invention provides a method to treat metabolic syndrome, comprising administering to a patient in need thereof, an effective amount of a compound of the formula: Y-Aib-EGTFTSDYSI-Aib-LDKIAQ-K((2-[2-(2-Amino-ethoxy)-ethoxy]-acetyl)ₐ-(γ-Glu)_{b}-CO—(CH₂)_{c}—CO₂H)-A-X₂₂-VQWLIAG-X₃₀
wherein
X₂₂ is F or 1-Nal;
X₃₀ is selected from the group consisting of G, GP, and GPSSG;
a is 1 or 2;
b is 1 or 2;
c is 16 or 18;
the C-terminal amino acid is optionally amidated as a C-terminal primary amide; or
a pharmaceutically acceptable salt thereof.

In an embodiment, present invention provides a method to treat obesity, comprising administering to a patient in need thereof, an effective amount of a compound of the formula: Y-Aib-EGTFTSDYSI-Aib-LDKIAQ-K((2-[2-(2-Amino-ethoxy)-ethoxy]-acetyl)ₐ-(γ-Glu)_{b}-CO—(CH₂)_{c}—CO₂H)-A-X₂₂-VQWLIAG-X₃₀
wherein
X₂₂ is F or 1-Nal;
X₃₀ is selected from the group consisting of G, GP, and GPSSG;
a is 1 or 2;
b is 1 or 2;
c is 16 or 18;
the C-terminal amino acid is optionally amidated as a C-terminal primary amide; or
a pharmaceutically acceptable salt thereof. In a further embodiment, the present invention provides a method to treat dyslipidemia, obesity, and/or hepatic steatosis associated with insulin resistance and diabetes, comprising administering to a patient in need thereof, an effective amount of a compound of the formula: Y-Aib-EGTFTSDYSI-Aib-LDKIAQ-K((2-[2-(2-Amino-ethoxy)-ethoxy]-acetyl)ₐ-(γ-Glu)_{b}-CO—(CH₂)_{c}—CO₂H)-A-X₂₂-VQWLIAG-X₃₀
wherein
X₂₂ is F or 1-Nal;
X₃₀ is selected from the group consisting of G, GP, and GPSSG;
a is 1 or 2;
b is 1 or 2;
c is 16 or 18;
the C-terminal amino acid is optionally amidated as a C-terminal primary amide; or
a pharmaceutically acceptable salt thereof. Additionally, the present invention provides a method to treat frailty or increase bone strength, comprising administering to a patient in need thereof, an effective amount of a compound of the formula: Y-Aib-EGTFTSDYSI-Aib-LDKIAQ-K((2-[2-(2-Amino-ethoxy)-ethoxy]-acetyl)ₐ-(γ-Glu)_{b}-CO—(CH₂)_{c}—CO₂H)-A-X₂₂-VQWLIAG-X₃₀
wherein
X₂₂ is F or 1-Nal;
X₃₀ is selected from the group consisting of G, GP, and GPSSG;
a is 1 or 2;
b is 1 or 2;
c is 16 or 18;
the C-terminal amino acid is optionally amidated as a C-terminal primary amide; or
a pharmaceutically acceptable salt thereof.

In an embodiment, the present invention provides a compound of the formula: Y-Aib-EGTFTSDYSI-Aib-LDKIAQ-K((2-[2-(2-Amino-ethoxy)-ethoxy]-acetyl)ₐ-(γ-Glu)_{b}-CO—(CH₂)_{c}—CO₂H)-A-X₂₂-VQWLIAG-X₃₀
wherein
X₂₂ is F or 1-Nal;
X₃₀ is selected from the group consisting of G, GP, and GPSSG;
a is 1 or 2;
b is 1 or 2;
c is 16 or 18;
the C-terminal amino acid is optionally amidated as a C-terminal primary amide; or
a pharmaceutically acceptable salt thereof; for use in therapy. In a further embodiment, the present invention provides a compound of the formula: Y-Aib-EGTFTSDYSI-Aib-LDKIAQ-K((2-[2-(2-Amino-ethoxy)-ethoxy]-acetyl)ₐ-(γ-Glu)_{b}-CO—(CH₂)_{c}—CO₂H)-A-X₂₂-VQWLIAG-X₃₀
wherein
X₂₂ is F or 1-Nal;
X₃₀ is selected from the group consisting of G, GP, and GPSSG;
a is 1 or 2;
b is 1 or 2;
c is 16 or 18;
the C-terminal amino acid is optionally amidated as a C-terminal primary amide; or
a pharmaceutically acceptable salt thereof; for use in the treatment of type 2 diabetes mellitus. In a further embodiment, the present invention provides a compound of the formula: Y-Aib-EGTFTSDYSI-Aib-LDKIAQ-K((2-[2-(2-Amino-ethoxy)-ethoxy]-acetyl)ₐ-(γ-Glu)_{b}-CO—(CH₂)_{c}—CO₂H)-A-X₂₂-VQWLIAG-X₃₀
wherein
X₂₂ is F or 1-Nal;
X₃₀ is selected from the group consisting of G, GP, and GPSSG;
a is 1 or 2;
b is 1 or 2;
c is 16 or 18;
the C-terminal amino acid is optionally amidated as a C-terminal primary amide; or
a pharmaceutically acceptable salt thereof; in simultaneous, separate, or sequential combination with one or more agents selected from metformin, thiazolidinediones, sulfonylureas, dipeptidyl peptidase 4 inhibitors, and sodium glucose co-transporters for use in the treatment of type 2 diabetes mellitus.

In a further embodiment, the present invention provides a compound of the formula: Y-Aib-EGTFTSDYSI-Aib-LDKIAQ-K((2-[2-(2-Amino-ethoxy)-ethoxy]-acetyl)ₐ-(γ-Glu)_{b}-CO—(CH₂)_{c}—CO₂H)-A-X₂₂-VQWLIAG-X₃₀
wherein
X₂₂ is F or 1-Nal;
X₃₀ is selected from the group consisting of G, GP, and GPSSG;
a is 1 or 2;
b is 1 or 2;
c is 16 or 18;
the C-terminal amino acid is optionally amidated as a C-terminal primary amide; or
a pharmaceutically acceptable salt thereof; in simultaneous, separate, or sequential combination with one or more agents selected from a GIP/GLP1 agonist, metformin, thiazolidinediones, sulfonylureas, dipeptidyl peptidase 4 inhibitors, and sodium glucose co-transporters for use in the treatment of type 2 diabetes mellitus. In an embodiment a GIP/GLP1 agonist is tirzepatide, or a pharmaceutical acceptable salt thereof.

In an embodiment, the present invention provides a compound of the formula: YXJEGTFTSDYSIX₂LDKIAQKA X₂₂ VQWLIAGGPSSGAPPPS
X₁ is Aib; and
X₂ is Aib;
X₂₂ is selected from the group consisting of F and 1-Nal;
or a pharmaceutically acceptable salt thereof.

In an embodiment, the present invention provides a compound of the formula: YXJEGTFTSDYSIX₂LDKIAQKAFVQWLIAGGPSSGAPPPS
X₁ is Aib; and
X₂ is Aib; or a
pharmaceutically acceptable salt thereof.

In an embodiment, the present invention provides a compound of the formula: YX₁EGTFTSDYSIX₂LDKIAQKAFVQWLIAGGPSSGAPPPS
X₁ is Aib;
X₂ is Aib; and
K of position 20 is chemically modified with time extension technology; or a pharmaceutically acceptable salt thereof.

In an embodiment, the present invention provides a compound of the formula: YX₁EGTFTSDYSIX₂LDKIAQKAFVQWLIAGGPSSGAPPPS
X₁ is Aib;
X₂ is Aib; and
the C-terminal amino acid is amidated as a C terminal primary amide;
or a pharmaceutically acceptable salt thereof.

In an embodiment, the present invention provides a compound of the formula: YX₁EGTFTSDYSIX₂LDKIAQKAX₂₂VQWLIAGGPSSGAPPPS
X₁ is Aib;
X₂ is Aib;
X₂₂ is selected from the group consisting of F and 1-Nal;
K of position 20 is optionally chemically modified with time extension technology; and
the C-terminal amino acid is optionally amidated as a C terminal primary amide; or a pharmaceutically acceptable salt thereof.

YXJEGTFTSDYSIX₂LDKIAQKA X₂₂VQWLIAG-X₃₀
wherein, X₁ is Aib;
X₂ is Aib;
X₂₂ is selected from the group consisting of F and 1-Nal;
and
X₃₀ is selected from the group consisting of G, GP, and GPSSG; or a pharmaceutically acceptable salt thereof.

In an embodiment, the present invention provides a compound of the formula: YX₁EGTFTSDYSIX₂LDKIAQKA X₂₂VQWLIAG-X₃₀
wherein, X₁ is Aib;
X₂ is Aib;
K of position 20 is chemically modified with time extension technology;
X₂₂ is selected from the group consisting of F and 1-Nal; and
X₃₀ is selected from the group consisting of G, GP, and GPSSG; or a
pharmaceutically acceptable salt thereof.

In an embodiment, the present invention provides a compound of the formula: YX1EGTFTSDYSIX₂LDKIAQKA X₂₂VQWLIAG-X₃₀
X₁ is Aib; and
X₂ is Aib;
K of position 20 is optionally chemically modified with time extension technology;
X₂₂ is selected from the group consisting of F and 1-Nal;
X₃₀ is selected from the group consisting of G, GP, and GPSSG;
the C-terminal amino acid is optionally amidated as a C terminal primary amide;
or a pharmaceutically acceptable salt thereof.

In an embodiment, the present invention provides a compound of the formula: YX₁EGTFTSDYSIX₂LDKIAQKAX₂₂ VQWLIAGGPSSGAPPPS
X₁ is Aib;
X₂ is Aib;
X₂₂ is selected from the group consisting of F and 1-Nal; and
the C-terminal amino acid is amidated as a C terminal primary amide; or a pharmaceutically acceptable salt thereof.

YX₁EGTFTSDYSIX₂LDKIAQKAX₂₂VQWLIAG-X₃₀
wherein, X₁ is Aib;
X₂ is Aib;
X₂₂ is selected from the group consisting of F and 1-Nal; and
X₃₀ is selected from the group consisting of G, GP, and GPSSG; or a pharmaceutically acceptable salt thereof.

In an embodiment, the present invention provides a compound of the formula:
YX₁EGTFTSDYSIX₂LDKIAQKAFVQWLIAG-X₃₀
wherein, X₁ is Aib;
X₂ is Aib;
K of position 20 is optionally chemically modified with time extension technology;
X₂₂ is selected from the group consisting of F and 1-Nal; and
X₃₀ is selected from the group consisting of G, GP, and GPSSG;
the C-terminal amino acid is optionally amidated as a C terminal primary amide; or a pharmaceutically acceptable salt thereof. In an embodiment, the present invention provides a compound of the present invention for use in glycemic control in adults with type 2 diabetes mellitus as an adjunct to diet and exercise. In an embodiment, the present invention provides a compound of the present invention for use in chronic weight management in adults with an initial body mass index ≥27 and type 2 diabetes mellitus as an adjunct to a reduced-calorie diet and increased physical activity.

In an embodiment, the present invention provides the use of a compound of the present invention for the manufacture of a medicament for the treatment of type 2 diabetes mellitus. In a further embodiment, the present invention provides the use of a compound of the present invention in simultaneous, separate, or sequential combination with one or more agents selected from metformin, thiazolidinediones, sulfonylureas, dipeptidyl peptidase 4 inhibitors, and sodium glucose co-transporters for the manufacture of a medicament for the treatment of type 2 diabetes mellitus. In a further embodiment, the present invention provides the use of a compound of the present invention in simultaneous, separate, or sequential combination with one or more agents selected from a GIP/GLP1 agonist, metformin, thiazolidinediones, sulfonylureas, dipeptidyl peptidase 4 inhibitors, and sodium glucose co-transporters for the manufacture of a medicament for the treatment of type 2 diabetes mellitus. In a further embodiment, the present invention provides the use of a compound of the present invention in simultaneous, separate, or sequential combination with one or more selected from a GIP/GLP1 agonist, metformin, thiazolidinediones, sulfonylureas, dipeptidyl peptidase 4 inhibitors, and sodium glucose co-transporters, wherein a GIP/GLP1 agonist is tirzepatide, or a pharmaceutically acceptable salt thereof, for the manufacture of a medicament for the treatment of type 2 diabetes mellitus.

In an embodiment of a compound disclosed and/or claimed herein, or a pharmaceutically acceptable salt thereof, the compound is an isotopic derivative of any one of the compounds described herein or a pharmaceutically acceptable salt thereof.

It is understood that the isotopic derivative can be prepared using any of a variety of art-recognized techniques. For example, the isotopic derivatives can generally be prepared by carrying out the procedures disclosed in the schemes and/or in the examples described herein or a pharmaceutically acceptable salt thereof, by substituting an isotopically containing reagent for a non-isotopically containing reagent.

In an embodiment of a compound disclosed and/or claimed herein, or a pharmaceutically acceptable salt thereof, the compound is a deuterium containing compound of any one of the compounds described herein and pharmaceutically acceptable salts thereof.

In the compounds of this invention any atom not specifically designated as a particular isotope is meant to represent any stable isotope of that atom. Unless otherwise stated, when an atom is designated specifically as “H” or “hydrogen”, the atom is understood to have hydrogen at its natural abundance isotopic composition. Also, unless otherwise stated, when an atom is designated specifically as “D” or “deuterium”, the atom is understood to have deuterium at an abundance substantially greater than the natural abundance of deuterium, which is 0.015%.

As used herein “time-extension technology” means a peptide time-extension technology for example, peptide conjugation to recombinant human serum albumin (“rHSA”), to a pharmaceutically acceptable polymer, such as polymeric sequence of amino acids (“XTEN”), unsulfated heparin-like carbohydrate polymer (“HEP”), hydroxyl ethyl starch (“HES”), llama heavy-chain antibody fragments (“VHH”), pegylation, Fc conjugation, bovine serum albumin (“BSA”) (Sleep, D. Epert Opin Drug Del (2015) 12, 793-812; Podust V N et. al. J Control. Release, 2015; ePUB; Hey, T. et. al. in: R. Kontermann (Ed.), Therapeutic Proteins: Strategies to Modulate their Plasma Half-Lives, Wiley-VCH Verlag Gmbh & Co. KGaA, Weinheim, Germany, 2012, pp 117-140; DeAngelis, P L, Drug Dev Delivery (2013) January 12/31/2012. In an embodiment time-extension technology is applied using a linker group. In an embodiment, the time-extension technology is applied using 0, 1, 2, or 3 amino acids as linker. In an embodiment, the time extension technology is an acylation other than ((2-[2-(2-Amino-ethoxy)-ethoxy]-acetyl)ₐ-(γ-Glu)_{b}-CO—(CH₂)_{c}—CO₂H), wherein a is 1 or 2; b is 1 or 2; and c is 16 or 18.

The present invention provides compounds that display selectivity for GIP and GLP-1 receptors versus receptors for glucagon and GLP-2. The term “selectivity” or “selective against” when used herein to reference GIP and GLP-1 activity in comparison to glucagon activity, refers to a compound that displays 1000-, 500-, or about 100-fold higher potency for GIP and GLP-1 over glucagon when the data is normalized from the respective in vitro binding assays. The term “selectivity” or “selective against” when used herein to reference GIP and GLP-1 activity in comparison to GLP-2 activity, refers to a compound that displays 250-, 200-, 100-, or about 50-fold higher potency for GIP and GLP-1 over GLP-2 when the data is normalized from the respective in vitro functional assays.

Fatty acids, through their albumin binding motifs, can improve the pharmacokinetics of a peptide by extending the half-life or by orienting the peptide for receptor activation, for example. In an embodiment, a peptide with no albumin binding motif may be selected to facilitate more frequent dosing and/or altered receptor binding and/or selectivity.

The present invention provides a method for treatment of type 2 diabetes in a patient comprising administering to a patient in need of such treatment an effective amount of a compound of the present invention, or a pharmaceutically acceptable salt thereof. The present invention also provides a method for treatment of type 2 diabetes in a patient comprising administering to a patient in need of such treatment an effective amount of a compound of the present invention, or a pharmaceutically acceptable salt thereof, wherein the administration is subcutaneous. The present invention also provides a method of treatment of type 2 diabetes in a patient comprising administering to a patient in need of such treatment an effective amount of a compound of the present invention, or a pharmaceutically acceptable salt thereof, and simultaneously, separately, or sequentially an effective amount of one or more other active ingredients. In one embodiment, the other active ingredient or ingredients is currently available oral glucose lowering drugs from a class of drugs that is considered prior to administration the standard of care as determined by industry guidelines such as the American Diabetes Association.

The present invention provides a method for treatment of chronic weight management in a patient comprising administering to a patient in need of such treatment an effective amount of a compound of the present invention, or a pharmaceutically acceptable salt thereof. The present invention also provides a method for treatment of chronic weight management in a patient comprising administering to a patient in need of such treatment an effective amount of a compound of the present invention, or a pharmaceutically acceptable salt thereof, wherein the administration is subcutaneous. The present invention also provides a method of treatment of chronic weight management in a patient comprising administering to a patient in need of such treatment an effective amount of a compound of the present invention, or a pharmaceutically acceptable salt thereof, and simultaneously, separately, or sequentially an effective amount of one or more other active ingredients.

The present invention provides a method for treatment of obesity in a patient comprising administering to a patient in need of such treatment an effective amount of a compound of the present invention, or a pharmaceutically acceptable salt thereof. The present invention also provides a method for treatment of obesity in a patient comprising administering to a patient in need of such treatment an effective amount of a compound of the present invention, or a pharmaceutically acceptable salt thereof, wherein the administration is subcutaneous. The present invention also provides a method of treatment of obesity in a patient comprising administering to a patient in need of such treatment an effective amount of a compound of the present invention, or a pharmaceutically acceptable salt thereof, and simultaneously, separately, or sequentially an effective amount of one or more other active ingredients.

The compounds of the present invention utilize a fatty acid chemically conjugated to the epsilon-amino group of a lysine side-chain. The fatty acid is conjugated to the epsilon-amino group of a lysine side-chain through a linker. The linker comprises [2-(2-Amino-ethoxy)-ethoxy]-acetyl)₂-(γGlu)ₐ wherein a is 1 to 2. Compounds of the present invention comprise a lysine at position 20 that is chemically modified through conjugation to the epsilon-amino group of the K side-chain with ([2-(2-Amino-ethoxy)-ethoxy]-acetyl)₂-(γGlu)ₐ-CO—(CH₂)_{b}—CO₂H wherein a is 1 to 2 and b is 10 to 20. As shown in the chemical structures of Example 1, the first unit of [2-(2-Amino-ethoxy)-ethoxy]-acetyl is linked to the epsilon-amino group of the lysine side-chain. The second unit of [2-(2-Amino-ethoxy)-ethoxy]-acetyl is then attached to the amino-group of the first unit of [2-(2-Amino-ethoxy)-ethoxy]-acetyl. Then, the first unit of γGlu is attached to the amino-group of the second unit of [2-(2-Amino-ethoxy)-ethoxy]-acetyl through the γ-carboxyl group of the side-chain. When a=2, the second unit of γGlu is attached to the α-amino-group of the first unit of γGlu through the γ-carboxyl group of the side-chain. Finally, the symmetrical fatty acid is attached to the α-amino—group of the first (when a=1) or second (when a=2) unit of γGlu.

The compounds of the invention are preferably formulated as pharmaceutical compositions administered by parenteral routes (e.g., subcutaneous, intravenous, intraperitoneal, intramuscular, or transdermal). Such pharmaceutical compositions and processes for preparing same are well known in the art. (See, e.g., Remington: The Science and Practice of Pharmacy (D. B. Troy, Editor, 21st Edition, Lippincott, Williams & Wilkins, 2006). The preferred route of administration is subcutaneous.

The compounds of the present invention may react with any of a number of inorganic and organic acids to form pharmaceutically acceptable acid addition salts. Pharmaceutically acceptable salts and common methodology for preparing them are well known in the art. See, e.g., P. Stahl, et al. Handbook of Pharmaceutical Salts: Properties, Selection and Use, 2nd Revised Edition (Wiley-VCH, 2011); S. M. Berge, et al., “Pharmaceutical Salts,” Journal of Pharmaceutical Sciences, Vol. 66, No. 1, January 1977. Pharmaceutically acceptable salts of the present invention include, but are not limited to, trifluoroacetate, hydrochloride, and acetate salts.

As used herein, the term “effective amount” refers to the amount or dose of compound of the present invention, or a pharmaceutically acceptable salt thereof which, upon single or multiple dose administration to the patient, provides the desired effect in the patient under diagnosis or treatment. An effective amount can be determined by the attending diagnostician, as one skilled in the art, by the use of known techniques and by observing results obtained under analogous circumstances. In determining the effective amount for a patient, a number of factors are considered by the attending diagnostician, including, but not limited to: the species of mammal; its size, age, and general health; the specific disease or disorder involved; the degree of or involvement or the severity of the disease or disorder; the response of the individual patient; the particular compound administered; the mode of administration; the bioavailability characteristics of the preparation administered; the dose regimen selected; the use of concomitant medication; and other relevant circumstances.

As used herein, the term “treating” or “to treat” includes restraining, slowing, stopping, or reversing the progression or severity of an existing symptom or disorder.

As used herein, “GIP/GLP1 agonist” means a treatment wherein the pharmacological activity of the treatment is an agonist of both GIP and GLP1 receptors. In an embodiment, a “GIP/GLP1 agonist” is a peptide. In an embodiment, “GIP/GLP1 agonist” is tirzepatide, or a pharmaceutically acceptable salt thereof.

Certain compounds of the present invention are generally effective over a wide dosage range. For example, dosages for once-weekly dosing may fall within the range of about 0.05 to about 30 mg per person per week. Certain compounds of the present invention may be dosed daily. Additionally, certain compounds of the present invention may be dosed once-weekly.

The amino acid sequences of the present invention contain the standard single letter or three letter codes for the twenty naturally occurring amino acids. Additionally, “Aib” is alpha amino isobutyric acid, and “1-Nal” is 1-Naphthylalanine.

The present invention also encompasses novel intermediates and processes useful for the synthesis of compounds of the present invention, or a pharmaceutically acceptable salt thereof. The intermediates and compounds of the present invention may be prepared by a variety of procedures known in the art. In particular, the process using chemical synthesis is illustrated in the Examples below. The specific synthetic steps for each of the routes described may be combined in different ways to prepare compounds of the present invention, or salts thereof. The reagents and starting materials are readily available to one of ordinary skill in the art. It is understood that these Examples are not intended to be limiting to the scope of the invention in any way.

### Example 1

### Y-Aib-EGTFTSDYSI-Aib-LDKIAQ-K((2-[2-(2-Amino-ethoxy)-ethoxy]-acetyl)₂-(γ-Glu)-CO—(CH₂)₁₈—CO₂H)-AFVQWLIAGGP-OH (SEQ ID NO:12)

The structure of SEQ ID NO: 12 is depicted below using the standard single letter amino acid codes, with the exception of residues Aib2, Aib13 and K₂O, where the structures of these amino acid residues have been expanded:

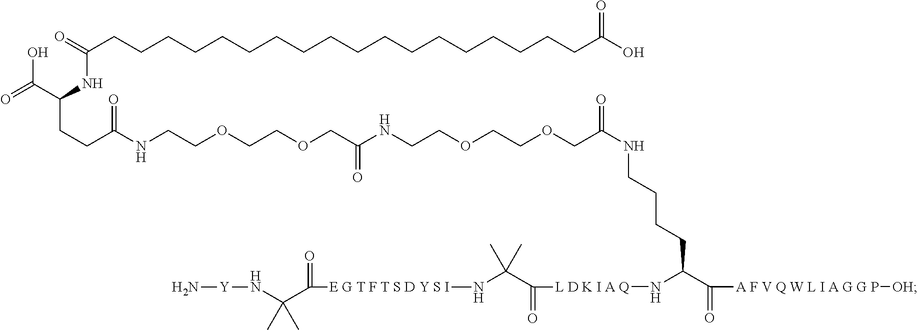

The peptide backbone of Example 1 is synthesized using Fluorenylmethyloxycarbonyl (Fmoc)/tert-Butyl (t-Bu) chemistry on a Symphony multiplex peptide synthesizer (Gyros Protein Technologies. Tucson, AZ).

The resin support consists of 1% DVB cross-linked polystyrene (H-Pro-2-chlorotrityl resin, 200-400 mesh, Novabiochem) at a substitution of 0.6 mmol/g. Standard side-chain protecting groups are used. Fmoc-Lys (Alloc)-OH is used for the lysine residue at position 20, and Boc-Tyr (tBu)-OH is used for the tyrosine residue at position 1. Fmoc groups are removed prior to each coupling step (2×10 minutes) using 20% piperidine in DMF. All standard amino acid couplings are performed for 1 hour, using an equal molar ratio of Fmoc amino acid (0.3 M in DMF), diisopropylcarbodiimide (0.9 M in DCM) and Oxyma (0.9 M in DMF), at a 9-fold molar excess over the theoretical peptide loading. Exceptions are couplings to Ca-methylated amino acids, which are coupled for 3 hours. After completion of the synthesis of the peptide backbone, the resin is thoroughly washed with DCM to remove residual DMF. The Alloc protecting group on the lysine residue at position 20 is selectively removed from the peptide resin by treatment with tetrakis(triphenylphosphine) palladium (0) (2-fold molar excess) and phenylsilane (30-fold molar excess) in DCM (3×30 min treatments), and the resin is thoroughly washed with sodium diethyldithiocarbamate (10% w/w in DMF, 3×5 min), followed by DCM and DMF.

Subsequent attachment of the linker moiety is accomplished by stepwise coupling of 2-[2-(2-Fmoc-amino-ethoxy)-ethoxy]-acetic acid (Fmoc-AEEA-OH, ChemPep, Inc.) and Fmoc-glutamic acid α-t-butyl ester (Fmoc-L-Glu-OtBu, Ark Pharm, Inc.), following the procedures described above for standard coupling and deprotection reactions. After removal of the final Fmoc protecting group, mono-OtBu-icosanedioic acid (WuXi AppTec, Shanghai, China) is coupled overnight using a 4-fold excess of the fatty acid, diisopropylcarbodiimide, and Oxyma (1:1:1 mol/mol/mol) in 1:1 DCM/DMF. After the synthesis is complete, the peptide-resin is washed with DCM and then thoroughly dried under vacuum.

The dry resin is treated with 10 mL of cleavage cocktail (trifluoroacetic acid: water: triisopropylsilane, 95:2.5:2.5 v/v) for 2 hours at room temperature. The resin is filtered off, washed twice each with 2 mL of neat TFA, and the combined filtrates are treated with 5-fold excess volume of cold diethyl ether (−20° C.) to precipitate the crude peptide. The peptide/ether suspension is then centrifuged at 3500 rpm for 2 min to form a solid pellet, the supernatant is decanted, and the solid pellet is triturated with ether two additional times and dried in vacuo. The crude peptide is solubilized in 20 mL of 20% acetonitrile/20% acetic acid/60% water and purified by RP-HPLC on a SymmetryPrep 7 μm C18 preparative column (19×300 mm, Waters) with linear gradients of a 100% acetonitrile and 0.1% TFA/water buffer system. The purity of peptide is assessed using analytical RP-HPLC and pooling criteria is >95%. Subsequent lyophilization of the final main product pool yielded the lyophilized peptide TFA salt. The main pool purity of Example 1 is found to be 96.0%. The molecular weight is determined by LC-MS (observed: M+3=1378.7; calculated M+3=1378.9).

### Assays

Provided below are the conditions and data for Examples in several assays. The provided assays support in vitro function and selectivity, immunogenicity profiling, pharmacokinetics, and in vivo type 2 diabetes models.

### Binding Assays

Glucagon (referred to as Gcg) is a Reference Standard prepared at Eli Lilly and Company. GLP-1, 7-36-NH₂ (referred to as GLP-1) is obtained from CPC Scientific (Sunnyvale, CA, 97.2% purity, 100 μM aliquots in 100% DMSO). GIP 1-42 (referred to as GIP) is prepared at Lilly Research Laboratories using peptide synthesis and HPLC chromatography as described above (>80% purity, 100 μM aliquots in 100% DMSO). [¹²⁵I]-radiolabeled Gcg, GLP-1, or GIP is prepared using [¹²⁵I]-lactoperoxidase and obtained from Perkin Elmer (Boston, MA).

Stably transfected cell lines were prepared by subcloning receptor cDNA into a pcDNA3 expression plasmid and transfected into human embryonic kidney (HEK) 293 (hGcgR and hGLP-1R) or Chinese Hamster Ovary (CHO) (hGIPR) cells followed by selection with Geneticin (hGLP-1R and hGIPR) or hygromycin B (hGcgR).

Two methods were used for the preparation of crude cell membranes.

Method 1: Frozen cell pellets were lysed on ice in hypotonic buffer containing 50 mM Tris HCl, pH 7.5, and Roche Complete™ Protease Inhibitors with EDTA. The cell suspension were disrupted using a glass Potter-Elvehjem homogenizer fitted with a Teflon® pestle for 25 strokes. The homogenate was centrifuged at about 4° C. at 1100× g for about 10 minutes. The supernatant was collected and stored on ice while the pellets were resuspended in homogenization buffer and rehomogenized, as described above. The homogenate was centrifuged at about 1100×g for about 10 minutes. The second supernatant was combined with the first supernatant and centrifuged at about 35000×g for about 1 hour at about 4° C. The resulting membrane pellet was resuspended in homogenization buffer containing protease inhibitors at approximately 1 to 3 mg/mL, quick frozen in liquid nitrogen and stored as aliquots in a−80° C. freezer until use.

Method 2: Frozen cell pellets were lysed on ice in hypotonic buffer containing 50 mM Tris HCl, pH 7.5, 1 mM MgCl₂, Roche Complete™ EDTA-free Protease Inhibitors and 25 units/ml DNAse I (Invitrogen). The cell suspension was disrupted using a glass Potter-Elvehjem homogenizer fitted with a Teflon® pestle for 20 to 25 strokes. The homogenate was centrifuged at about 4° C. at about 1800×g for about 15 minutes. The supernatant was collected and stored on ice while the pellets were resuspended in homogenization buffer (without DNAse I) and rehomogenized, as described above. The homogenate was centrifuged at about 1800×g for about 15 minutes. The second supernatant was combined with the first supernatant and centrifuged an additional time at about 1800×g for about 15 minutes. The overall supernatant was then centrifuged at about 25000×g for about 30 minutes at about 4° C. The resulting membrane pellet was resuspended in homogenization buffer (without DNAse I) containing protease inhibitors at approximately 1 to 3 mg/mL and stored as aliquots in a−80° C. freezer until use.

### Binding Determination Methods

The equilibrium binding dissociation constants (K_{d}) for the various receptor/radioligand interactions were determined from homologous competition binding analysis instead of saturation binding due to high propanol content in the [¹²⁵I] stock material. The K_{d} values determined for the receptor preparations were as follows: hGcgR (3.9 nM), hGLP-1R (1.2 nM) and hGIPR (0.14 nM).

### [¹²⁵I]-Glucagon Binding

The human Gcg receptor binding assays were performed using a Scintillation Proximity Assay (SPA) format with wheat germ agglutinin (WGA) beads (Perkin Elmer). The binding buffer contained 25 mM 4-(2-hydroxyethyl)-1-piperazineethanesulfonic acid (HEPES), pH 7.4, 2.5 mM CaCl₂), 1 mM MgCl₂, 0.1% (w/v) bacitracin (Research Products), 0.003% (w/v) Polyoxyethylenesorbitan monolaurate (TWEEN®-20), and Roche Complete™ Protease Inhibitors without EDTA. Peptides and Gcg were thawed and 3-fold serially diluted in 100% DMSO (10 point concentration response curves). Next, 5 L serially diluted compound or DMSO was transferred into Corning® 3632 clear bottom assay plates containing 45 μL assay binding buffer or unlabeled Gcg control (non-specific binding or NSB, at 1 μM final). Then, 50 μL [¹²⁵I]-Gcg (0.15 nM final), 50 μL human GcgR membranes (1.5 μg/well) and 50 μL of WGA SPA beads (80 to 150 μg/well) were added with a Biotek Multiflo dispenser. Plates were sealed and mixed on a plate shaker (setting 6) for 1 minute and read with a PerkinElmer Trilux MicroBeta® scintillation counter after about 12 hours of incubation/settling time at room temperature. Final assay concentration ranges for peptides tested in response curves was typically 1150 nM to 0.058 nM and for the control Gcg from 1000 nM to 0.05 nM.

### [¹²⁵I]-GLP-1 Binding

The human GLP-1 receptor binding assay was performed using an SPA format with WGA beads. The binding buffer contained 25 mM HEPES, pH 7.4, 2.5 mM CaCl₂, 1 mM MgCl₂, 0.1% (w/v) bacitracin, 0.003% (w/v) TWEEN®-20, and Roche Complete™ Protease Inhibitors without EDTA. Peptides and GLP-1 were thawed and 3-fold serially diluted in 100% DMSO (10 point concentration response curves). Next, 5 μL serially diluted compound or DMSO was transferred into Corning® 3632 clear bottom assay plates containing 45 μL assay binding buffer or unlabeled GLP-1 control (non-specific binding or NSB, at 0.25 μM final). Then, 50 μL [¹²⁵I]-GLP-1 (0.15 nM final), 50 μL human GLP-1R membranes (0.5 μg/well and 50 μL of WGA SPA beads (100 to 150 μg/well) were added with a Biotek Multiflo dispenser. Plates were sealed and mixed on a plate shaker (setting 6) for 1 minute and read with a PerkinElmer Trilux MicroBeta® scintillation counter after 5 to 12 hours of incubation/settling time at room temperature. Final assay concentration ranges for peptides tested in response curves were typically 1150 nM to 0.058 nM and for the control GLP-1, 250 nM to 0.013 nM.

### [¹²⁵I]-GIP Binding

The human GIP receptor binding assay was performed using an SPA format with WGA beads. The binding buffer contained 25 mM HEPES, pH 7.4, 2.5 mM CaCl₂, 1 mM MgCl₂, 0.1% (w/v) bacitracin, 0.003% (w/v) TWEEN®-20, and Roche Complete™ Protease Inhibitors without EDTA. Peptides and GIP were thawed and 3 fold serially diluted in 100% DMSO (10 point concentration response curves). Next, 5 L serially diluted compound or DMSO was transferred into Corning® 3632 clear bottom assay plates containing 45 μL assay binding buffer or unlabeled GIP control (non-specific binding or NSB, at 0.25 μM final). Then, 50 μL [¹²⁵I]-GIP (0.075-0.15 nM final), 50 μL human GIPR membranes (3 μg/well) and 50 μL of WGA SPA beads (100 to 150 μg/well) were added with a Biotek Multiflo dispenser. Plates were sealed and mixed on a plate shaker (setting 6) for 1 minute and read with a PerkinElmer Trilux MicroBeta® scintillation counter after 2.5 to 12 hours of incubation/settling time at room temperature. Final assay concentration ranges for peptides tested in response curves was typically 1150 to 0.058 nM or 115 nM to 0.0058 nM and for the control GIP, 250 nM to 0.013 nM.

### Binding Assay Data Analysis

Raw CPM data for concentration curves of peptides, Gcg, GLP-1, or GIP were converted to percent inhibition by subtracting nonspecific binding (binding in the presence of excess unlabeled Gcg, GLP-1, or GIP, respectively) from the individual CPM values and dividing by the total binding signal, also corrected by subtracting nonspecific binding. Data were analyzed using four-parameter (curve maximum, curve minimum, IC₅₀, Hill slope) nonlinear regression routines (Genedata Screener, version 12.0.4, Genedata AG, Basal, Switzerland). The affinity constant (Kᵢ) was calculated from the absolute IC₅₀ value based upon the equation Kᵢ=IC₅₀/(1+D/K_{d}) where D is the concentration of radioligand used in the experiment, IC₅₀ is the concentration causing 50% inhibition of binding and K_{d} is the equilibrium binding dissociation constant of the radioligand (described above). Values for Kᵢ were reported as the geometric mean, with error expressed as the standard error of the mean (SEM) and n is equal to the number of independent replicates (determined in assays performed on different days). Geometric Means were calculated as follows:

Geometric Mean=10^{(Arithmetic Mean of Log Ki Values))}

n=1/x means that only one value out of the total number of replicates (x) is used to express the mean. SEM is only calculated when n=2 or greater non-qualified results exist. Means are expressed as GeoMetric means with the standard error of the mean (SEM) and the number of replicates (n) indicated in parenthesis.

TABLE 1
In vitro Binding Affinity (Kᵢ) of indicated Examples and
comparator molecules for human GLP-1R, GcgR and GIPR.
Binding Affinity

| Molecule or | Kᵢ, nM (SEM, n) | | |
|---|---|---|---|
| Example | hGIPR | hGLP-1R | hGCGR |
| hGIP | 0.135 | | |
| (1-42)NH₂ | (0.00585, | | |
| | 378/380) | | |
| hGLP-1 | | 0.791 | |
| (7-36)NH₂ | | (0.0236, 545) | |
| hGCG | | | 3.07 |
| | | | (0.115, |
| | | | 510/511) |
| 1 | 0.320 | 10.9 | >240 |
| | (0.161, n = 2) | (2.26, n = 2) | (n = 1/2) |

The in vitro binding data support the selective GIP/GLP-1 receptor binding of the Example compound.

Functional hGIP-R, hGLP-1R and hGCGR Assays

Methods: Functional activity was determined using cAMP formation in HEK-293 clonal cell lines expressing hGIPR, hGLP-1R or hGCGR. hGIPR, hGLP-1R or hGCGR receptor-expressing cells were treated with a control polypeptide or one of Examples (20 point concentration-response curve in DMSO, 2.75-fold Labcyte Echo direct dilution, 384 well plate Corning Cat #3570) in DMEM (Gibco Cat #31053) supplemented with 1× GlutaMAX™ (Gibco Cat #35050), 0.1% bovine casein (Sigma C4765-10ML), 250 μM IBMX (3-Isobutyl-1-methylxanthine, Acros Cat #228420010) and 20 mM HEPES (Gibco Cat #15630) in a 20 μL assay volume (final DMSO concentration was 0.5%). Experiments also were performed under substantially identical assay conditions with the addition of 1.0% fatty acid free, globulin free human serum albumin (Sigma Cat #A3782).

After a 30-min incubation at 37° C., the resulting increase in intracellular cAMP was quantitatively determined using a CisBio CAMP Dynamic 2 HTRF Assay Kit (62AM4PEJ). Briefly, cAMP levels within the cell are detected by adding the CAMP-d2 conjugate in cell lysis buffer (10 μL) followed by the antibody anti-cAMP-Eu³⁺-Cryptate, also in cell lysis buffer (10 μL). The resulting competitive assay was incubated for at least 60 min at room temperature, and then was detected using a PerkinElmer Envision® instrument with excitation at 320 nm and emission at 665 nm and 620 nm. Envision units (emission at 665 nm/620 nm*10,000) are inversely proportional to the amount of cAMP present and are converted to nM cAMP per well using a CAMP standard curve. The amount of cAMP generated (nM) in each well is converted to a percent of the maximal response observed with human GIP(1-42)NH₂, hGLP-1(7-36)NH₂ or hGCG. A relative EC₅₀ value and percent top (Eₘₐₓ) were derived by non-linear regression analysis using the percent maximal response vs. the concentration of peptide added, fitted to a four-parameter logistic equation.

Results: Functional data for hGIP(1-42)NH₂, hGLP-1(7-36)NH₂, hGCG and the Example compound are provided below in Table 2 (0.1% bovine casein) and Table 3 (0.1% bovine casein, 1.0% human serum albumin).

TABLE 2
Functional cAMP Potency (EC₅₀) and Efficacy (Eₘₐₓ) for Peptides
Incubated at 37 °C (in the presence of 0.1% bovine casein).

| | hGIPR ^{a} | | hGLP-1R ^{a} | | hGCGR ^{a} | |
|---|---|---|---|---|---|---|
| | EC₅₀, nM | Emax, % ± | EC₅₀, nM | Emax, % ± | EC₅₀, nM | Emax, % |
| Example | (SEM, n) ^{b} | SEM ^{c} | (SEM, n) ^{b} | SEM ^{c} | (SEM, n) ^{b} | (SEM, n) ^{c} |
| hGIP(1- | 0.988 | 100 ± | | | | |
| 42)NH₂ | (0.0675, | 0.863 | | | | |
| | 244/246) | | | | | |
| hGLP- | | | 0.315 | 106 ± | | |
| 1(7- | | | (0.00999, | 0.687 | | |
| 36)NH₂ | | | 400) | | | |
| hGCG | | | | | 1.95 | 101 |
| | | | | | (0.0802, | (1.13, |
| | | | | | 301) | n = 301) |
| 1 | 0.589 | | 39.1 | | >9950 | |
| | (0.168, | | (8.58, | | (n = 1/3) | |
| | n = 3) | | n = 2) | | | |

ND = Not Determined
^{a} Expression density is determined using homologous competition binding of [¹²⁵I]GLP-1(7-36)NH₂ at hGLP-1R (112 fmol/mg protein), [¹²⁵I]GCG at hGCGR (98 fmol/mg protein) and [¹²⁵I]GIP(1-42) at hGIPR (124 fmol/mg protein).
^{b} EC₅₀, nM = the Geometric Mean, followed by the Standard Error of the Mean and the number of observations in parenthesis.
^{c} Eₘₐₓ, % = the Arithmetic Mean ± the Standard Error of the Mean for the percent of maximal response to GLP-1(7-36)NH₂ at hGLP-1R, GCG at hGCGR or GIP(1-42)NH₂ at hGIPR.
All values shown are to three (3) significant digits.

TABLE 3
Functional cAMP Potency (EC₅₀) and Efficacy (Eₘₐₓ) for Peptides Incubated at
37 °C (in the presence of 0.1% bovine casein and 1.0% human serum albumin).

| | hGIPR ^{a} | | hGLP-1R ^{a} | | hGCGR ^{a} | |
|---|---|---|---|---|---|---|
| | EC₅₀, nM | Emax, % ± | EC₅₀, nM | Emax, % ± | EC₅₀, nM | Emax, % ± |
| Example | (SEM, n) ^{b} | SEM ^{c} | (SEM, n) ^{b} | SEM ^{c} | (SEM, n) ^{b} | SEM ^{c} |
| hGIP(1- | 0.597 | 98.0 ± | | | | |
| 42)NH₂ | (0.0522, | 1.16 | | | | |
| | 132) | | | | | |
| hGLP- | | | 0.340 | 105 ± | | |
| 1(7- | | | (0.0195, | 1.39 | | |
| 36)NH₂ | | | 133) | | | |
| hGCG | | | | | 2.37 | 102 |
| | | | | | (0.153, | (se = 1.49, |
| | | | | | 135) | n = 135) |
| 1 | 60.0 | | 6560 | | >9950 | |
| | (4.44, | | (213, | | (n = 1/3) | |
| | n = 3) | | n = 3) | | | |

ND = Not Determined
^{a} Expression density is determined using homologous competition binding of [¹²⁵I]GLP-1(7-36)NH₂ at hGLP-1R (112 fmol/mg protein), [¹²⁵I]GCG at hGCGR (98 fmol/mg protein) and [¹²⁵I]GIP(1-42) at hGIPR (124 fmol/mg protein).
^{b} EC₅₀, nM = the Geometric Mean, followed by the Standard Error of the Mean and the number of observations in parenthesis.
^{c} Eₘₐₓ, % = the Arithmetic Mean ± the Standard Error of the Mean for the percent of maximal response to GLP-1(7-36)NH₂ at hGLP-1R, GCG at hGCGR or GIP(1-42)NH₂ at hGIPR.
All values shown are to three (3) significant digits.

### In Vivo Studies

### Pharmacokinetics in Male CD-1 Mice

The pharmacokinetics of a test peptide is evaluated following a single subcutaneous administration of 200 nMol/kg to male CD-1 mice. Blood samples are collected over 168 hours and resulting individual plasma concentrations are used to calculate pharmacokinetic parameters. Plasma (K₃EDTA) concentrations are determined using a qualified LC/MS method that measures the intact mass of the test peptide. Each test peptide and an analog as an internal standard are extracted from 100% mouse plasma using immunoaffinity based precipitation with anti-GIP/GLP1 antibodies. Instruments are combined for LC/MS detection. Mean pharmacokinetic parameters determine if the test peptide is consistent with an extended pharmacokinetic profile.

### Insulin Secretion in Male Wistar Rats

Male Wistar rats with femoral artery and femoral vein cannulas (Envigo, Indianapolis, IN) (280-320 grams) are single-housed in polycarbonate cages with filter tops. Rats maintained on a 12:12 h light-dark cycle (lights on at 6:00 A.M.) at 21° C. and receive food and deionized water ad libitum. Rats are randomized by body weight and dosed 1.5 ml/kg s.c. at test peptide doses of 0.04, 0.1, 0.3, 1, 3, and 10 nmol/kg 16 hours prior to glucose administration then fasted. Animals are weighed and anesthetized with sodium pentobarbital dosed i.p. (65 mg/kg, 30 mg/ml). A time 0 blood sample is collected into EDTA tubes after which glucose is administered i.v. (0.5 mg/kg, 5 ml/kg). Blood samples are collected for glucose and insulin levels at time 2, 4, 6, 10, 20 and 30 min post intravenous administration of glucose. Plasma glucose levels are determined using a clinical chemistry analyzer. Plasma insulin is determined using an electrochemiluminescence assay (Meso Scale, Gaithersburg, MD). Glucose and insulin AUC are examined compared to the vehicle control with n=5 animals per group. Results are presented (SEM) (N). Results show the test peptide effect on insulin secretion during intravenous glucose tolerance test. Results show test peptide dose dependent effect on insulin secretion.

### Diet-Induced Obese C57/B16 Mice

C57/B16 diet-induced obese (DIO) male mice (Taconic, Germantown, NY) weighing 41-50 g are used. Animals are individually housed in a temperature-controlled (24° C.) facility with a 12 hour light/dark photoperiod (lights off at 10:00 AM and lights on at 10:00 PM), with free access to food and water. After 2 week acclimatization to the facility, mice are randomized to treatment groups (n=6/group) based on body weight so each group has similar starting mean body weight.

Mice are treated with either vehicle (40 mM Tris-HCl at pH 8.0) or a test peptide between the dose range of about 0.03 nmol/kg to about 10 nmol/kg. Treatments are subcutaneously administered to ad libitum fed DIO mice 30-90 minutes prior to the onset of the dark cycle daily (QD) for 14 days. Monitor body weight and food intake daily.

Data are expressed as mean±SEM of 5-6 rats per group. Statistical analyses are assessed by one-way ANOVA followed by Dunnett's multiple comparison test to compare treatment groups to vehicle group or each other. Significant differences are identified at p<0.05.

$$Percent Body Weight = \frac{Body weight after 14 - day treatment}{Body weight before treatment started} \times 100$$

“0” dose group represents the vehicle-treated mice during each study. All data are expressed as mean±SEM of 5-6 mice per group. “Δ from vehicle” refers to difference between body weight at day 15 between test and vehicle groups. “% change” refers to percent decrease in body weight between days 1 and 15 in test groups. Record percent decrease in body weight for animals receiving vehicle. The Δ from vehicle and % change data are statistically significantly different (p<0.05) than control for a peptide testing positive in the assay.

### Amino Acid Sequences

(Human GIP)
SEQ ID NO: 1
YAEGTFISDYSIAMDKIHQQDFVNWLLAQKGKKNDWKHNITQ-NH₂
(Human GLP-1)
SEQ ID NO: 2
HAEGTFTSDVSSYLEGQAAKEFIAWLVKGR-NH₂
SEQ ID NO: 3
YX₁EGTFTSDYSIX₂LDKIAQKAFVQWLIAGGPSSGAPPPS

wherein X₁ is Aib; X₂ is Aib; K at position 20 is chemically modified through conjugation to the epsilon-amino group of the K side-chain with ([2-(2-Amino-ethoxy)-ethoxy]-acetyl)₂-(γGlu) 1-CO—(CH₂)₁₈—CO₂H; and the C-terminal amino acid is amidated as a C-terminal primary amide

(Human Glucagon)
SEQ ID NO: 4
HSQGTFTSDYSKYLDSRRAQDFVQWLMNT
SEQ ID NO: 5
GPSSG
SEQ ID NO: 6
Y-Aib-EGTFTSDYSI-Aib-LDKIAQ-K((2-[2-(2-Amino-
ethoxy)-ethoxy]-acetyl)ₐ-(γ-Glu)_{b}-CO-(CH₂)_{c}-
CO₂H)-A-X₂₂-VQWLIAG-X₃₀
SEQ ID NO: 7
Y-Aib-EGTFTSDYSI-Aib-LDKIAQ-K((2-[2-(2-Amino-
ethoxy)-ethoxy]-acetyl)ₐ-(γ-Glu)_{b}-CO-(CH₂)_{c}-
CO₂H)-A-X₂₂-VQWLIAG-X₃₀
SEQ ID NO: 8
Y-Aib-EGTFTSDYSI-Aib-LDKIAQ-K((2-[2-(2-Amino-
ethoxy)-ethoxy]-acetyl)₂-(γ-Glu)-CO-(CH₂)₁₈-
CO₂H)-AFVQWLIAGG-NH₂
SEQ ID NO: 9
Y-Aib-EGTFTSDYSI-Aib-LDKIAQ-K((2-[2-(2-Amino-
ethoxy)-ethoxy]-acetyl)₂-(γ-Glu)-CO-(CH₂)₁₈-
CO₂H)-AFVQWLIAGG-OH
SEQ ID NO: 10
Y-Aib-EGTFTSDYSI-Aib-LDKIAQ-K((2-[2-(2-Amino-
ethoxy)-ethoxy]-acetyl)₂-(γ-Glu)-CO-(CH₂)₁₈-
CO₂H)-AFVQWLIAGGPSSG-NH₂
SEQ ID NO: 11
Y-Aib-EGTFTSDYSI-Aib-LDKIAQ-K((2-[2-(2-Amino-
ethoxy)-ethoxy]-acetyl)₂-(γ-Glu)-CO-(CH₂)₁₈-
CO₂H)-AFVQWLIAGGPSSG-OH
SEQ ID NO: 12
Y-Aib-EGTFTSDYSI-Aib-LDKIAQ-K((2-[2-(2-Amino-
ethoxy)-ethoxy]-acetyl)₂-(γ-Glu)-CO-(CH₂)₁₈-
CO₂H)-AFVQWLIAGGP-OH
SEQ ID NO: 13
Y-Aib-EGTFTSDYSI-Aib-LDKIAQ-K((2-[2-(2-Amino-
ethoxy)-ethoxy]-acetyl)ₐ-(γ-Glu)_{b}-CO-(CH₂)_{c}-
CO₂H)-A-X₂₂-VQWLIAG-X₃₀
SEQ ID NO: 14
Y-Aib-EGTFTSDYSI-Aib-LDKIAQ-K((2-[2-(2-Amino-
ethoxy)-ethoxy]-acetyl)ₐ-(γ-Glu)_{b}-CO-(CH₂)_{c}-
CO₂H)-A-X₂₂-VQWLIAG-X₃₀
SEQ ID NO: 15
GPSSG-NH₂

We claim:

1. A pharmaceutical composition comprising a compound of the formula: Y-Aib-EGTFTSDYSI-Aib-LDKIAQ-K((2-[2-(2-Amino-ethoxy)-ethoxy]-acetyl)ₐ-(γ-Glu)_{b}-CO—(CH₂)_{c}—CO₂H)-A-X₂₂-VQWLIAG-X₃₀
wherein
X₂₂ is F or 1-Nal;
X₃₀ is selected from the group consisting of G, GP, and GPSSG;
a is 1 or 2;
b is 1 or 2;
c is 16 or 18;
the C-terminal amino acid is optionally amidated as a C-terminal primary amide (SEQ ID NO: 6); or
a pharmaceutically acceptable salt thereof.

2. The composition as claimed by claim 1 wherein c is 18.

3. The composition as claimed by claim 2,
comprising a compound selected from the group consisting of Y-Aib-EGTFTSDYSI-Aib-LDKIAQ-K((2-[2-(2-Amino-ethoxy)-ethoxy]-acetyl)₂-(ΔGlu)-CO—(CH₂)₁₈—CO₂H)-AFVQWLIAGG-NH₂;
Y-Aib-EGTFTSDYSI-Aib-LDKIAQ-K((2-[2-(2-Amino-ethoxy)-ethoxy]-acetyl)₂-(γ-Glu)-CO—(CH₂)₁₈—CO₂H)-AFVQWLIAGG-OH;
Y-Aib-EGTFTSDYSI-Aib-LDKIAQ-K((2-[2-(2-Amino-ethoxy)-ethoxy]-acetyl)₂-(γ-Glu)-CO—(CH₂)₁₈—CO₂H)-AFVQWLIAGGPSSG-NH₂;
Y-Aib-EGTFTSDYSI-Aib-LDKIAQ-K((2-[2-(2-Amino-ethoxy)-ethoxy]-acetyl)₂-(γ-Glu)-CO—(CH₂)₁₈—CO₂H)-AFVQWLIAGGPSSG-OH; and
Y-Aib-EGTFTSDYSI-Aib-LDKIAQ-K((2-[2-(2-Amino-ethoxy)-ethoxy]-acetyl)₂-(γ-Glu)-CO—(CH₂)₁₈—CO₂H)-AFVQWLIAGGP-OH, or a pharmaceutically acceptable salt thereof.

4. The composition as claimed by claim 1 comprising a compound that is Y-Aib-EGTFTSDYSI-Aib-LDKIAQ-K((2-[2-(2-Amino-ethoxy)-ethoxy]-acetyl)₂-(γ-Glu)-CO—(CH₂)₁₈—CO₂H)-AFVQWLIAGGP-OH; or a pharmaceutically acceptable salt thereof.

5. The composition as claimed by claim 1 comprising a compound of the Formula: 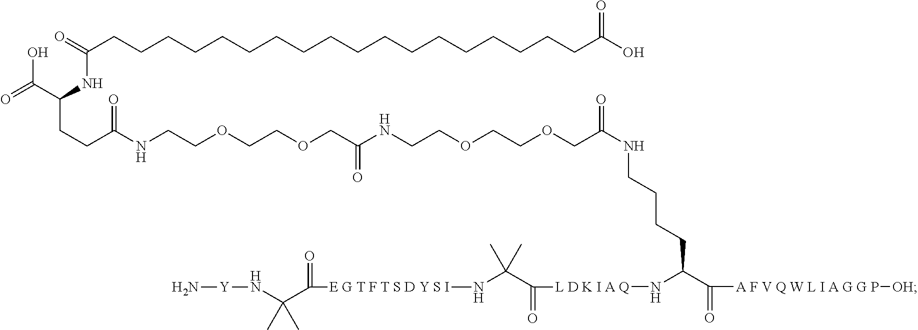
or a pharmaceutically acceptable salt thereof.

6. The composition as claimed by claim 1 further comprising at least one pharmaceutically acceptable excipient.

7. A method for treating type 2 diabetes in a patient in need thereof, comprising administering an effective amount of a composition as claimed by claim 1.

8. A method for treating type 2 diabetes in a patient in need thereof, comprising administering an effective amount of a composition as claimed by claim 1, in simultaneous, separate, or sequential combination with one or more agents selected from a GIP/GLP1 agonist, metformin, thiazolidinediones, sulfonylureas, dipeptidyl peptidase 4 inhibitors, and sodium glucose co-transporter.

9. A method as claimed by claim 8 wherein the GIP/GLP1 agonist is tirzepatide, or a pharmaceutically acceptable salt thereof.

10. A method for treating obesity in a patient in need thereof, comprising administering an effective amount of a composition as claimed by claim 1.

11. A method for chronic weight management in a patient in need thereof, comprising administering an effective amount of a composition as claimed by claim 1.

12. (canceled)

13. (canceled)

14. A method for improving glycemic control in a patient in need thereof, comprising administering an effective amount of a composition as claimed by claim 1.

15. A compound of the formula: Y-Aib-EGTFTSDYSI-Aib-LDKIAQ-K((2-[2-(2-Amino-ethoxy)-ethoxy]-acetyl)ₐ-(γ-Glu)_{b}-CO—(CH₂)_{c}—CO₂H)-A-X₂₂-VQWLIAG-X₃₀
wherein
X₂₂ is F or 1-Nal;
X₃₀ is selected from the group consisting of G, GP, and GPSSG;
a is 1 or 2;
b is 1 or 2;
c is 16 or 18;
the C-terminal amino acid is optionally amidated as a C-terminal primary amide; or
a pharmaceutically acceptable salt thereof; for use in therapy.

16. A compound of the formula: Y-Aib-EGTFTSDYSI-Aib-LDKIAQ-K((2-[2-(2-Amino-ethoxy)-ethoxy]-acetyl)ₐ-(γ-Glu)_{b}-CO—(CH₂)_{c}—CO₂H)-A-X₂₂-VQWLIAG-X₃₀
wherein
X₂₂ is F or 1-Nal;
X₃₀ is selected from the group consisting of G, GP, and GPSSG;
a is 1 or 2;
b is 1 or 2;
c is 16 or 18;
the C-terminal amino acid is optionally amidated as a C-terminal primary amide; or
a pharmaceutically acceptable salt thereof; for use in a composition for treating type 2 diabetes.

17. A compound of the formula: Y-Aib-EGTFTSDYSI-Aib-LDKIAQ-K((2-[2-(2-Amino-ethoxy)-ethoxy]-acetyl)ₐ-(γ-Glu)_{b}-CO—(CH₂)_{c}—CO₂H)-A-X₂₂-VQWLIAG-X₃₀
wherein
X₂₂ is F or 1-Nal;
X₃₀ is selected from the group consisting of G, GP, and GPSSG;
a is 1 or 2;
b is 1 or 2;
c is 16 or 18;
the C-terminal amino acid is optionally amidated as a C-terminal primary amide; or
a pharmaceutically acceptable salt thereof; for use in improving chronic weight management.

18. A compound of the formula: Y-Aib-EGTFTSDYSI-Aib-LDKIAQ-K((2-[2-(2-Amino-ethoxy)-ethoxy]-acetyl)ₐ-(γ-Glu)_{b}-CO—(CH₂)_{c}—CO₂H)-A-X₂₂-VQWLIAG-X₃₀
wherein
X₂₂ is F or 1-Nal;
X₃₀ is selected from the group consisting of G-NH₂, G-OH, GP-OH, GPSSG-NH₂ (SEQ ID NO:15) and GPSSG-OH (SEQ ID NO:5);
a is 1 or 2;
b is 1 or 2;
c is 16 or 18;
the C-terminal amino acid is optionally amidated as a C-terminal primary amide; or
a pharmaceutically acceptable salt thereof.

19. A compound as claimed by claim 18 wherein c is 18.

20. A compound as claimed by claim 18 wherein the compound is selected from the group consisting of:
Y-Aib-EGTFTSDYSI-Aib-LDKIAQ-K((2-[2-(2-Amino-ethoxy)-ethoxy]-acetyl)₂-(γ-Glu)-CO—(CH₂)₁₈—CO₂H)-AFVQWLIAGGP-NH₂
Y-Aib-EGTFTSDYSI-Aib-LDKIAQ-K((2-[2-(2-Amino-ethoxy)-ethoxy]-acetyl)₂γ-Glu)-CO—(CH₂)₁₈—CO₂H)-AFVQWLIAGG-NH₂
Y-Aib-EGTFTSDYSI-Aib-LDKIAQ-K((2-[2-(2-Amino-ethoxy)-ethoxy]-acetyl)₂-(γ-Glu)-CO—(CH₂)₁₈—CO₂H)-AFVQWLIAGG-OH
Y-Aib-EGTFTSDYSI-Aib-LDKIAQ-K((2-[2-(2-Amino-ethoxy)-ethoxy]-acetyl)₂-(γ-Glu)-CO—(CH₂)₁₈—CO₂H)-AFVQWLIAGGPSSG-NH₂
Y-Aib-EGTFTSDYSI-Aib-LDKIAQ-K((2-[2-(2-Amino-ethoxy)-ethoxy]-acetyl)₂-(γ-Glu)-CO—(CH₂)₁₈—CO₂H)-AFVQWLIAGGPSSG-OH; and
Y-Aib-EGTFTSDYSI-Aib-LDKIAQ-K((2-[2-(2-Amino-ethoxy)-ethoxy]-acetyl)₂-(γ-Glu)-CO—(CH₂)₁₈—CO₂H)-AFVQWLIAGGP-OH; or
a pharmaceutically acceptable salt thereof.

21. A compound of the formula:
Y-Aib-EGTFTSDYSI-Aib-LDKIAQ-K-A-X₂₂-VQWLIAG-X₃₀
wherein
X₂₂ is F or 1-Nal;
X₃₀ is selected from the group consisting of G, GP, and GPSSG; and
the C-terminal amino acid is optionally amidated as a C-terminal primary amide; or a pharmaceutically acceptable salt thereof.

22. A compound as claimed by claim 21 wherein X₃₀ is GP; or a pharmaceutically acceptable salt thereof.

23. A compound as claimed by claim 22 wherein at least one hydrogen is deuterated to contain greater than 0.015% deuterium.

24. A composition comprising a compound as claimed by claim 21.

25. A composition as claimed by claim 24 comprising at least one pharmaceutically acceptable excipient.

26. A method for treating type 2 diabetes in a patient in need thereof, comprising administering an effective of amount of a compound as claimed by claim 21, or a pharmaceutically acceptable salt thereof.

27. A method for treating obesity in a patient in need thereof comprising administering an effective amount of a compound as claimed by claim 21, or a pharmaceutically acceptable salt thereof.


SEQUENCE LISTING (15 sequences)

SEQ ID NO: 1 - protein, 42 aa, Homo sapiens
  MOD_RES 42: note = Amidation
YAEGTFISDYSIAMDKIHQQDFVNWLLAQKGKKNDWKHNITQ

SEQ ID NO: 2 - protein, 30 aa, Homo sapiens
  MOD_RES 30: note = Amidation
HAEGTFTSDVSSYLEGQAAKEFIAWLVKGR

SEQ ID NO: 3 - protein, 39 aa, synthetic construct
  SITE 2: note = X at position 2 is Aib
  SITE 13: note = X at position 13 is Aib
  MOD_RES 20: note = K at position 20 is chemically modified through conjugation to the epsilon-amino group of the K side-chain with (2-[2-(2-Amino-ethoxy)-ethoxy]-acetyl)2-(gamma-Glu)1- CO-(CH2)18-CO2H
  MOD_RES 39: note = C-terminal amino acid is amidated as a C-terminal primary amide
YXEGTFTSDYSIXLDKIAQKAFVQWLIAGGPSSGAPPPS

SEQ ID NO: 4 - protein, 29 aa, Homo sapiens
HSQGTFTSDYSKYLDSRRAQDFVQWLMNT

SEQ ID NO: 5 - protein, 5 aa, synthetic construct
GPSSG

SEQ ID NO: 6 - protein, 30 aa, synthetic construct
  SITE 2: note = X at position 2 is Aib
  SITE 13: note = X at position 13 is Aib
  SITE 22: note = X at position 22 is F or 1-Nal
  SITE 30: note = X at position 30 G-NH2, G-OH, GP-OH, GPSSG-NH2 (SEQ ID NO:15) and GPSSG-OH (SEQ ID NO:5)
  MOD_RES 20: note = K at position 20 is chemically modified through conjugation to the epsilon-amino group of the K side-chain with (2-[2-(2-Amino-ethoxy)-ethoxy]-acetyl)a-(gamma-Glu)b- CO-(CH2)c-CO2H; a=1 or 2, b=1 or 2, c=16 or 18
  MOD_RES 30: note = C-terminal amino acid is optionally amidated as a C-terminal primary amide
YXEGTFTSDYSIXLDKIAQKAXVQWLIAGX

SEQ ID NO: 7 - protein, 30 aa, synthetic construct; identical to SEQ ID NO: 6
  SITE 2: note = X at position 2 is Aib
  SITE 13: note = X at position 13 is Aib
  MOD_RES 20: note = K at position 20 is chemically modified through conjugation to the epsilon-amino group of the K side-chain with (2-[2-(2-Amino-ethoxy)-ethoxy]-acetyl)a-(gamma-Glu)b- CO-(CH2)c-CO2H; a = 1 or 2, b = 1 or 2, c = 16 or 18
  SITE 22: note = X at position 22 is F or 1-Nal
  SITE 30: note = X at position 30 is G-NH2, G-OH, GP-OH, GPSSG-NH2 (SEQ ID NO:15) and GPSSG-OH (SEQ ID NO:5)

SEQ ID NO: 8 - protein, 30 aa, synthetic construct
  SITE 2: note = X at position 2 is Aib
  SITE 13: note = X at position 13 is Aib
  MOD_RES 20: note = K at position 20 is chemically modified through conjugation to the epsilon-amino group of the K side-chain with (2-[2-(2-Amino-ethoxy)-ethoxy]-acetyl)2-(gamma-Glu)-C O-(CH2)18-CO2H
  MOD_RES 30: note = C-terminal amino acid is amidated as a C-terminal primary amide.
YXEGTFTSDYSIXLDKIAQKAFVQWLIAGG

SEQ ID NO: 9 - protein, 30 aa, synthetic construct; identical to SEQ ID NO: 8
  SITE 2: note = X at position 2 is Aib
  SITE 13: note = X at position 13 is Aib
  MOD_RES 20: note = K at position 20 is chemically modified through conjugation to the epsilon-amino group of the K side-chain with (2-[2-(2-Amino-ethoxy)-ethoxy]-acetyl)2-(gamma-Glu)-C O-(CH2)18-CO2H

SEQ ID NO: 10 - protein, 34 aa, synthetic construct
  SITE 2: note = X at position 2 is Aib
  SITE 13: note = X at position 13 is Aib
  MOD_RES 20: note = K at position 20 is chemically modified through conjugation to the epsilon-amino group of the K side-chain with (2-[2-(2-Amino-ethoxy)-ethoxy]-acetyl)2-(gamma-Glu)-C O-(CH2)18-CO2H
  MOD_RES 34: note = C-terminal amino acid is amidated as a C-terminal primary amide.
YXEGTFTSDYSIXLDKIAQKAFVQWLIAGGPSSG

SEQ ID NO: 11 - protein, 34 aa, synthetic construct; identical to SEQ ID NO: 10
  SITE 2: note = X at position 2 is Aib
  SITE 13: note = X at position 13 is Aib
  MOD_RES 20: note = K at position 20 is chemically modified through conjugation to the epsilon-amino group of the K side-chain with (2-[2-(2-Amino-ethoxy)-ethoxy]-acetyl)2-(gamma-Glu)-C O-(CH2)18-CO2H

SEQ ID NO: 12 - protein, 31 aa, synthetic construct
  SITE 2: note = X at position 2 is Aib
  SITE 13: note = X at position 13 is Aib
  MOD_RES 20: note = K at position 20 is chemically modified through conjugation to the epsilon-amino group of the K side-chain with (2-[2-(2-Amino-ethoxy)-ethoxy]-acetyl)2-(gamma-Glu)-C O-(CH2)18-CO2H
YXEGTFTSDYSIXLDKIAQKAFVQWLIAGGP

SEQ ID NO: 13 - protein, 30 aa, synthetic construct; identical to SEQ ID NO: 6
  SITE 2: note = X at position 2 is Aib
  SITE 13: note = X at position 13 is Aib
  MOD_RES 20: note = K at position 20 is chemically modified through conjugation to the epsilon-amino group of the K side-chain with (2-[2-(2-Amino-ethoxy)-ethoxy]-acetyl)a-(gamma-Glu)b- CO-(CH2)c-CO2H; a = 1 or 2, b = 1 or 2, c = 16 or 18
  SITE 22: note = X at position 22 is F or 1-Nal
  MOD_RES 30: note = the C-terminal amino acid is optionally amidated as a C-terminal primary amide
  SITE 30: note = X at position 30 is G, GP, or GPSSG (SEQ ID NO:5)

SEQ ID NO: 14 - protein, 30 aa, synthetic construct; identical to SEQ ID NO: 6
  SITE 2: note = X at position 2 is Aib
  SITE 13: note = X at position 13 is Aib
  MOD_RES 20: note = K at position 20 is chemically modified through conjugation to the epsilon-amino group of the K side-chain with (2-[2-(2-Amino-ethoxy)-ethoxy]-acetyl)a-(gamma-Glu)b- CO-(CH2)c-CO2H; a = 1 or 2; b = 1 or 2; c = 16 or 18
  SITE 22: note = X at position F or 1-Nal
  SITE 30: note = X at position 30 is G, GP, or GPSSG (SEQ ID NO:5)

SEQ ID NO: 15 - protein, 5 aa, synthetic construct
  MOD_RES 5: note = C-terminal amino acid is amidated as a C-terminal primary amide
GPSSG